(19) United States
(12) Patent Application Publication
(10) Pub. No.: US 2021/0346601 A1
(43) Pub. Date: Nov. 11, 2021

(54) INTERVENTIONAL DOSING SYSTEMS AND METHODS

(71) Applicant: AMGEN INC., Thousand Oaks, CA (US)

(72) Inventors: Heejin Lee, Bedford, MA (US); Scott Robert Gibson, Granada Hills, CA (US); Mehran Mojarrad, Thousand Oaks, CA (US); Jeffrey Schacherl, Thousand Oaks, CA (US)

(21) Appl. No.: 17/264,139

(22) PCT Filed: Sep. 23, 2019
(86) PCT No.: PCT/US19/52359

§ 371 Date: Jan. 28, 2021

Related U.S. Application Data
(60) Provisional application No. 62/735,476, filed on Sep. 24, 2018.

(51) Int. Cl. A61M 5/172; A61M 5/14; A61M 5/168

(52) U.S. Cl. CPC A61M 5/1723; A61M 5/1407; A61M 5/14248; A61M 2205/18; A61M 5/16827

(57) ABSTRACT

Systems and methods for interventional dosing based on sensed feedback are disclosed. A system may include one or more reservoirs filled or fillable with, respectively, one or more drug products, an administration member, a fluid delivery system, one or more sensors, and optionally a controller. The administration member may be insertable into a patient and connected or connectable in fluid communication with the one or more reservoirs. The fluid delivery system may be operable to deliver the one or more drug products from the one or more reservoirs to the patient via the administration member. The one or more sensors may be operable to sense one or more biological conditions of the patient, including, but not limited to, a cytokine level and/or a biomarker indicative of cytokine release syndrome. The controller may be configured to control operation of the fluid delivery system based on output from the one or more sensors.

### CROSS-REFERENCE TO RELATED APPLICATION

Priority is claimed to U.S. Provisional Patent Application No. 62/735,476, filed Sep. 24, 2018, the entire contents of which are hereby incorporated by reference.

### FIELD OF DISCLOSURE

The present disclosure generally relates to drug delivery systems and methods. More particularly, the present disclosure relates to interventional dosing techniques responsive to changes in a monitored condition of a patient.

### BACKGROUND

Drugs are administered to treat a variety of conditions and diseases. Use of certain drugs can have unintended side effects, including ones that are adverse to the health of the patient. It is not always predictable whether a patient will have an adverse reaction to an administered drug. As a consequence, depending on the severity of the potential side effect(s) it may be necessary to monitor the condition of the patient during and/or after drug administration. If the patient does happen to experience adverse side effect(s), administration of the drug may be suspended or dose adjusted and/or another drug may be administered in an effort to counteract the side effect. The efficacy of such interventional dosing measures depends on early detection of the side effects, as well as timely administration of the counteractive drug.

As described in more detail below, the present disclosure sets forth systems and methods for patient monitoring and interventional dosing techniques embodying advantageous alternatives to existing systems and methods, and that may address one or more of the challenges or needs mentioned herein, as well as provide other benefits and advantages.

### SUMMARY

One aspect of the present disclosure provides a system including one or more reservoirs filled or fillable with, respectively, one or more drug products, an administration member, a fluid delivery system, one or more sensors, and optionally a controller. The one or more reservoirs may include a first reservoir filled or fillable with a first drug product. The administration member may be insertable into a patient and connected or connectable in fluid communication with the first reservoir. The fluid delivery system may be operable to deliver the first drug product from the first reservoir to the patient via the administration member. The one or more sensors may be operable to sense one or more biological conditions of the patient. The controller may be configured to control operation of the fluid delivery system based on output from the one or more sensors. In some embodiments, the controller may be configured to operate the fluid delivery system to suspend, terminate, or throttle delivery of the first drug product to the patient based on: a first biological condition sensed by the one or more sensors and/or a second biological condition sensed by the one or more sensors. Furthermore, in some embodiments, the one or more reservoirs may include a second reservoir filled or fillable with a second drug product that is stored separate from the first drug product; and the controller may be configured to operate the fluid delivery system to initiate delivery of the second drug product to the patient based on: the first biological condition sensed by the one or more sensors and/or the second biological condition sensed by the one or more sensors. Furthermore, in some embodiments, the second drug product may include a therapeutic agent for treating a condition or syndrome induced by administration of the first drug product.

Another aspect of the present disclosure provides a method including: (a) operating a fluid delivery system to deliver a first drug product from a reservoir to a patient via an administration member; (b) sensing, via one or more sensors, one or more biological conditions of the patient while, before, and/or after the first drug product is being delivered; and optionally (c) operating, automatically via a controller or manually, the fluid delivery system to suspend, terminate, or throttle delivery of the first drug product to the patient based on the one or more sensed biological conditions. The method may additionally include: (d) operating, automatically via the controller or manually, the fluid delivery system to initiate delivery of a second drug product from a second reservoir to the patient based on the one or more sensed biological conditions. In some embodiments, the second drug product may include a therapeutic agent for treating or managing a condition or syndrome induced by administration of the first drug product.

### BRIEF DESCRIPTION OF THE DRAWINGS

It is believed that the disclosure will be more fully understood from the following description taken in conjunction with the accompanying drawings. Some of the drawings may have been simplified by the omission of selected elements for the purpose of more clearly showing other elements. Such omissions of elements in some drawings are not necessarily indicative of the presence or absence of particular elements in any of the exemplary embodiments, except as may be explicitly delineated in the corresponding written description. Also, none of the drawings is necessarily to scale.

FIG. 1 is a schematic diagram of a drug delivery system according to an embodiment of the present disclosure.

FIG. 2 is a block diagram of a method of operating a drug delivery system, such as the drug delivery system illustrated in FIG. 1, according to an embodiment of the present disclosure.

FIG. 3 is a cross-sectional view of an embodiment of a drug delivery system including an on-body injector.

### DETAILED DESCRIPTION

The present disclosure generally relates to closed-loop drug delivery and biosensing systems and methods for monitoring the condition of a patient undergoing a drug therapy and providing interventional dosing in the event that the patient experiences an adverse side effect to the drug therapy. The systems and methods disclosed herein are particularly well suited for patients at risk of developing Cytokines Release Syndrome (CRS) as a consequence of a drug therapy, although they also have uses outside of this particular application.

CRS is a form of systemic inflammatory response that arises as an adverse effect after patients receive immunotherapy agents. It can occur following T cell engaging therapies including, for example, bispecific T cell engaging (e.g., BiTE®) antibody constructs such as blinatumomab (e.g., BLINCYTO®) and chimeric antigen receptor (CAR) T cell receptors. CRS can be life-threatening, and potentially fatal in certain cases. Hospitalization is therefore recommended post immunotherapy treatment, so patients can be carefully monitored by a healthcare professional for symptoms of CRS. Even if the patient is not hospitalized, he or she may be instructed to remain close to the location where the immunotherapy treatment was received, sometimes for a period of four or more weeks, so that urgent medical care can be provided should there be indications of CRS. Additionally, the patient may need to be monitored at least once daily for a week at a certified healthcare facility for signs of CRS following the immunotherapy treatment. Such monitoring can be burden for both patients and healthcare providers.

Antipyretics and/or intravenous fluids can be administered to help manage CRS. In addition or as an alternative, an anti-cytokine agent may be administered, such as a corticosteroid (e.g., dexamethasone) and/or an anti-interleukin-6 (IL-6) receptor antibody (e.g., tocilizumab). The efficacy of such anti-cytokine treatments can depend on their administration at an early stage of CRS. Thus, identifying the first warning signs of CRS and taking prompt medical action can be important.

Anti-cytokine agents and other drug products for treating CRS usually take the form of an injectable fluid. Patients who are uncomfortable with or do not have the necessary training to perform a self-injection may have to return to the hospital or other medical facility to receive the injection. Considering that the patient may be in a weakened state from cancer or other disease, requiring the patient to travel back to the hospital or other medical facility can be a significant burden.

The present disclosure describes systems and methods having various automated or semi-automated features or steps to assist with identifying the onset of CRS or another syndrome or condition induced by the current or previous administration of a drug product, as well as facilitating various interventional dosing measures including, but not limited to, the self-administration of a drug product for treating the induced syndrome or condition. The presently disclosed systems and methods advantageously facilitate early detection of adverse side effects such as CRS and provide for timely and appropriate medical intervention. The burden on healthcare providers to perform periodic blood tests and/or other patient monitoring to detect adverse side effects is therefore reduced. Moreover, patients can monitor and/or mitigate adverse side effects without having to visit an intensive management center such as a hospital. Instead, such tasks can be performed at a self-administration site including, for example, a managed care site, a wellness clinic, or the patient's home.

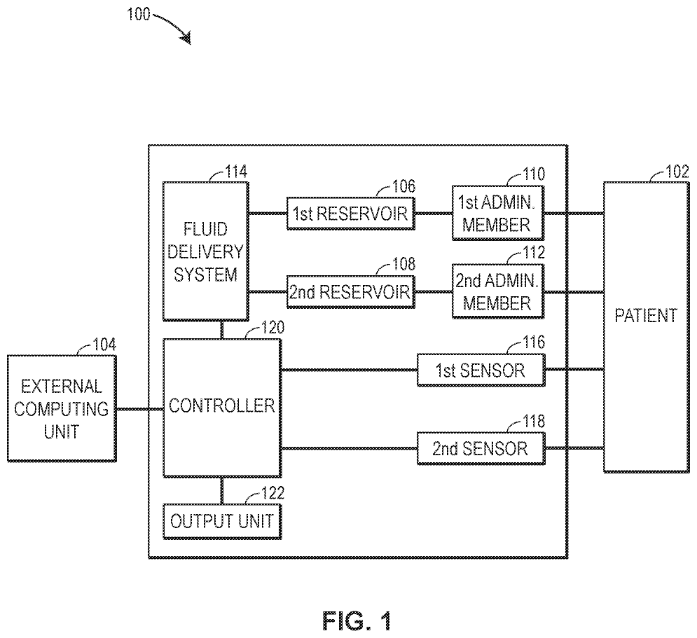

FIG. 1 illustrates a drug delivery system 100 according to an embodiment of the disclosure. The drug delivery system 100 may be associated with a patient 102, who may use the drug delivery system 100 to inject or infuse one or more drug products as part of a therapeutic regimen. The drug delivery system 100 may communicate information with an external computing device 104 (e.g., a smartphone, smartwatch, desktop computer, server, etc.) via one or more intermediate computing devices and/or one or more networks. In turn, the external computing device 104 may communicate with the patient 102 and/or one or more other computing devices and their associated parties (e.g., a healthcare provider such as a doctor or caregiver) directly or indirectly via one or more intermediate computing devices and/or one or more networks.

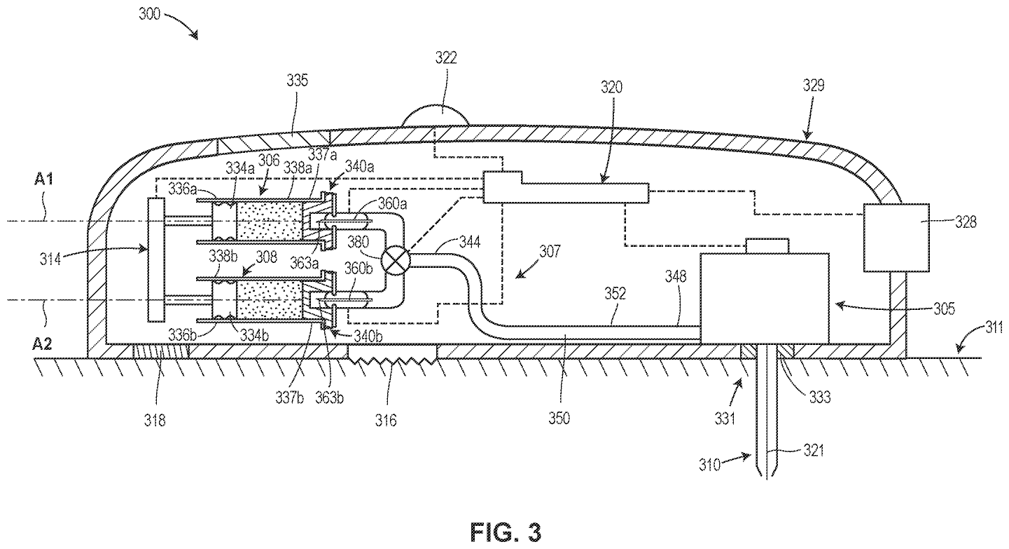

In some embodiments, the drug delivery system 100 may be defined by a plurality of discrete components assembled with each other at the time of use. In one such embodiment, the drug delivery system 100 may include a bedside infusion pump or other stationary or non-ambulatory infusion pump which is connected at the time of use to one or more drug reservoirs, which in turn are connected at the time of use to the patient via one or more tubing sets and one or more needles. In alternative embodiments, some or all of the components of drug delivery system 100 may be pre-assembled and/or contained within a single housing or unit. In such alternative embodiments, the drug delivery system 100 may be formed as: (i) a wearable injector, such as the skin-attachable on-body injector described below in connection with FIG. 3 or a clothing-attachable ambulatory infusion pump designed to deliver the drug product via flexible tubing and an infusion set external to a pump housing; or (ii) a hand-held injector, such as an autoinjector. In still further alternative embodiments, the drug delivery system 100 may be formed by any combination of: a stationary infusion pump (e.g., a bedside infusion pump), a wearable injector, an autoinjector, and/or a conventional manually-operated syringe.

In some embodiments the drug delivery system 100 may be portable such that it can be carried or worn by a patient before, after, and/or during drug delivery; whereas, in other embodiments, the drug delivery system 100 may be remain stationary over the duration of drug delivery.

The drug delivery system 100 may utilize one or more routes of administration depending on the volume, duration, and/or type of drug to be administered, among other considerations. Such routes of administration include, but are not limited to, intravenous, intra-arterial, subcutaneous, transdermal, intradermal, intramuscular, intrathecal, intracerebral, epidural, intraocular, nasal, inhalation, oral, and/or topical. In embodiments where the fluid system 100 is configured to deliver or assist with delivering two or more drug products to the patient, different administration routes may be utilized for some or all of the drug products, including any combination of the routes of administration mentioned herein, or other routes of administration.

The drug delivery system 100 may include one or more reservoirs filled (e.g., pre-filled) or fillable (e.g., filled at the time of use of the drug delivery system 100) respectively with one or more drug products, which may also be referred to herein as medicaments or medications. In embodiments where multiple reservoirs are included, each reservoir may separately store a respective drug product so that the drug products are not allowed to mix prior to use. The drug product may be, but is not limited to, various biologicals such as peptides, peptibodies, or antibodies. The drug product may be in a fluid or liquid form, although the disclosure is not limited to a particular state. According to some embodiments, the reservoir(s) may each be defined by rigid-walled cylinder having an internal bore, such as a syringe, vial, or cartridge. In other embodiments, the reservoir(s) may each be defined by a non-rigid collapsible pouch, such as an IV bag.

In some embodiments, the drug delivery system 100 may have an integrated reconstitution subsystem onboard to dilute a lyophilized drug into a liquid form. In certain such embodiments, a diluent reservoir may be included for storing a diluent solution and a lyophilized reservoir may be included storing a lyophilized compound separate from the diluent solution. Furthermore, a fluid drive mechanism may be included for mixing the diluent solution in the diluent reservoir with the lyophilized compound in the lyophilized reservoir. In some embodiments, the fluid drive mechanism may transfer the diluent solution from the diluent reservoir into the lyophilized reservoir and/or provide any circulation and/or agitation needed to achieve full reconstitution. In some embodiments, an additional final reconstituted drug reservoir may be included and serve as a delivery reservoir from which the reconstituted drug is discharged into the patient; whereas, in other embodiments, the lyophilized reservoir may serve as the delivery reservoir. While the reconstitution subsystem may be physically integrated into the drug delivery system 100 in certain embodiments, in other embodiments the reconstitution subsystem may constitute a separate unit which is in fluid communication with the drug delivery system 100. Having a separate unit may simplify the reconstitution process for healthcare providers in certain cases.

In the embodiment illustrated in FIG. 1, the drug delivery system 100 includes a first reservoir 106 filled or fillable with a first drug product, and a second reservoir 108 filled or fillable with a second drug product. In some embodiments, the first and second reservoirs 106 and 108 may be mechanically interconnected (e.g., as part of a single reservoir assembly) and potentially immovable relative to each other over the course of drug delivery; whereas, in other embodiments, the first and second reservoirs 106 and 108 may be separate from each other and thus free to move independently of each other. In embodiments where the first drug product is a lyophilized drug, a third reservoir may be included for storing a diluent solution as described above.

In some embodiments, the second drug product in the second reservoir 108 may be a therapeutic agent for treating or managing a condition or syndrome induced by administration of the first drug product from the first reservoir 106. In certain such embodiments, the first drug product in the first reservoir 106 may include an immunotherapy agent including, but not limited to, a bispecific T cell engaging (e.g., BiTE®) antibody constructs (e.g., blinatumomab) and/or a chimeric antigen receptor (CAR) T cell receptor (e.g., an anti-CD19 CAR-T cell); and the second drug product in the second reservoir 106 may include a therapeutic agent for treating or managing any potential CRS induced by administration of the immunotherapy agent, wherein such a therapeutic agent includes, but is not limited to, an antipyretic, an anti-cytokine agent (e.g., dexamethasone, methylprednisolone, or other corticosteroid), an anti-interleukin-6 (IL-6) receptor antibody (e.g., tocilizumab), and/or an anti-IL-6 chimeric monoclonal antibody (e.g., siltuximab).

The drug delivery system 100 may include one or more administration members for establishing fluid or another kind of communication between the one or more reservoirs and the patient 102. In some embodiments, each administration member may have a first end connected or connectable in fluid communication with a respective reservoir and a second end to be inserted into the patient 102. In some embodiments, the second end may have a sharpness sufficient to penetrate at least through the patient's skin and into subcutaneous tissue, a vein, an artery, other anatomical structure. In some embodiments, each administration member may include a cannula. The cannula may include a rigid or semi-rigid needle or blunt cannula, or may be in a flexible form, by example and not by way of limitation. The cannula may be integrated with the other elements of the drug delivery system 100, or the cannula may be separate from the other elements of the drug delivery system 100 until immediately prior to use. According to certain embodiments, the drug delivery system 100 may further include an inserter or introducer member to introduce the second end of the cannula into the patient, although this is not required according to each embodiment of the disclosure. The introducer member may, in certain embodiments, be withdrawn back into a housing of the drug delivery system 100, thereby leaving the cannula in the patient 102. In such embodiments, the cannula may be constructed of a relatively flexible or soft material such as plastic, whereas the introducer member, which may be a solid or hollow needle or trocar, may be constructed a relatively rigid or hard material such as metal. In other embodiments, the cannula may part of an infusion set to facilitate intravenous administration and may be connected in fluid communication with the one or more reservoirs via flexible tubing. In certain such embodiments, the introducer element may be an external applicator or trocar device and the drug delivery system 100 may be a wearable, ambulatory, or standalone infusion system.

FIG. 1 illustrates an embodiment of the drug delivery system 100 including a first administration member 110 and a second administration member 112. The first administration member 110 has a first end connected or connectable in fluid communication with the first reservoir 106, and a second end to be inserted into the patient 102. Similarly, the second first administration member 112 has a first end connected or connectable in fluid communication with the second reservoir 108, and a second end to be inserted into the patient 102. In some embodiments, the second administration member 112 may be omitted, and the first end of the first administration member 110 may be selectively connected in fluid communication with the first and second reservoirs 106 and 108 one at a time via, e.g., a controllable valve member.

Still referring to FIG. 1, the drug delivery system 100 may include a fluid delivery system 114 operable to deliver the first drug product from the first reservoir 106 to the patient via the first administration member 110 and/or deliver the second drug product from the second reservoir 108 to the patient via the second administration member 112. The fluid delivery system 114 may store the actuation energy and/or provide the motive force needed to expel the first and/or second drug products from their respective reservoirs 106 and/or 108. In some embodiments, the fluid delivery system 114 is powered by an external energy source such as a battery and/or other electric power supply. In other embodiments, the fluid delivery system 114 may itself store the actuation energy. The fluid delivery system 114 may include a pump (e.g., a peristaltic pump), an electric-motor-driven plunger, a spring-driven plunger (utilizing, e.g., a helical compression spring, a helical extension spring, a helical torsion spring, a spiral torsion spring, etc.), osmotically-driven force or pressure on a plunger, a source of pressurized and releasable gas or liquid, and a swellable gel, an inflatable or balloon-type reservoir having elastic walls which store potential energy when the reservoir is filled with a drug and which collapse inwardly for discharging the drug when a valve or flow path is opened, or any combination thereof. Furthermore, the fluid delivery system 114 may be controllable to actuate the first reservoir 106 independently of the second reservoir 108, and vice versa. In alternative embodiments, multiple fluid delivery systems may be included such that each reservoir can be actuated by its own respective fluid delivery system.

The fluid delivery system 114 may be operated to deliver the first and/or second drug product continuously to the patient at a specified rate over a specified period of time (e.g., 10 mL per hour for a duration of 24 hours, 5 mL per hour for a duration of 48 hours, or 0.6 mL per hour for a duration of 7 days) in accordance with a dosing regimen and/or interventional dosing regimen. The rate of delivery may depend on various factors including, but not limited to, a patient's weight, a patient's body surface, physiological factors such as patient's core body temperature and severity of reaction to certain drugs, and/or the medical advice of a healthcare provider. In other embodiments, the fluid delivery system 114 may be operated to deliver the entire volume of the first and/or second drug product to the patient as a single bolus over a relatively short period of time (e.g., several seconds, several tens of a seconds, several minutes, several tens of minutes, an hour, or several hours).

With continued reference to FIG. 1, the drug delivery system 100 may include one or more sensors operable to sense one or more biological conditions of the patient. The one or more sensors may operate continuously over the duration of drug delivery and provide real-time measurements of the biological condition(s) of the patient. The sensed biological condition(s) may include, for example, a level or change in level of a biochemical or analyte. In embodiments where the potential for CRS is being monitored, the biochemical may include a cytokine, chemokine, and/or other biomarker indicative of CRS. Such biomarkers include without limitation: interleukin-1 (IL-1) alpha, IL-1 beta, IL-1 receptor antagonist (IL-1RA), IL-2, IL-3, IL-4, IL-5, IL-6, soluble IL-6 receptor (sIL-6R), IL-7, IL-8, IL-8 (HA), IL-10, IL-13, IL-12p70, IL-12/IL-23 p40, IL-15, IL-16, IL-17A, IL-18, IL-22, granulocyte-colony stimulating factor (G-CSF), granulocyte-macrophage colony-stimulating factor (GM-CSF), interferon gamma (IFN-gamma), tumor necrosis factor alpha (TNF-alpha), TNF-beta, vascular endothelial growth factor A (VEGF-A), brain-derived neurotrophic factor (BDNF), IP-10, eotaxin, eotaxin-3, monocyte chemoattractant protein-1 (MCP-1), MCP-4, macrophage-derived chemokine (MDC), macrophage inflammatory protein-1 alpha (MIP-1 alpha), MIP-1 beta, soluble gp130 (sgp130), ferritin, and C-reactive protein (CRP). In some embodiments, the one or more sensors may collect or sample a biofluid having the biochemical of interest. Such biofluids include, for example: blood, blood plasma, blood serum, intracellular fluid, intravascular fluid, interstitial fluid, sweet (e.g., eccrine sweat), saliva, tears, urine, and nasal mucosa, or any combination thereof. In addition to or as an alternative to sensing a biochemical(s), the one or more sensors may be operable to detect or sense one or more other biological conditions of the patient including, for example, body temperature (e.g., skin temperature, core body temperature, etc.), respiration rate, heart rate, blood pressure, blood oxygen level, and blood oxygen satuation, or any combination thereof.

In order to the sense the biological condition(s) of interest the one or more sensors may utilize any suitable sensing pathway including, for example, those which are or involve: electrical (e.g., conductivity, etc.), chemical, electrochemical, mechanical (e.g., force), electromechanical, amperometric, potentiometric, piezoelectric, optical (e.g., Raman spectroscopy, infrared spectroscopy, near infrared (NIR) spectroscopy, mid infrared (MIR) spectroscopy, etc.), electrochemiluminescence (e.g., use of fluorophores or chromophores), field-effect transistor-based biosensing (BioFET) (e.g., immunoFET, DNA-FET, multicolor FRET (mFRET), enzyme field effect transistor, cell-potential FET, beetle/chip FET, etc.), Forster Resonance Energy Transfer (FRET), multicolor FREt (mFRET), acoustic (e.g., ultrasound sensor, etc.), vibrational, thermometric (e.g., thermocouple, etc.), fluid pressure, accelerometer, and biometric (e.g., fingerprint, voice, etc.), or any combination thereof.

In some embodiments, the one or more sensors may include a probe including, for example, a microneedle, an array of microneedles, a conventional needle (e.g., a syringe needle), a soft cannula, a sweat collector (involving, e.g., passive sweat collection, active sweat collection via reverse iontophoresis, active sweat collection via cholinergic sweat gland secretory stimulating compounds, etc.), and optical instrument (e.g., camera, interferometer, photometer, polarimeter, reflectometer, refractometer, spectrometer, monochromator, autocollimator, surface plasmon resonance-based instruments, etc.), or any combination thereof. In embodiments where the one or more sensors includes an optical instrument, an artificial light source may also be included for illuminating or interrogating the biochemical of interest. In some embodiments, the probe may be temporarily inserted into or implanted in the patient's tissue, whereas in other embodiments the probe may be disposed at the skin surface or slightly above the skin surface. In a preferred embodiment, the one or more sensors include a probe that non-invasive or minimally-invasive, although invasive-type sensors are not excluded by the present disclosure. Furthermore, in some embodiments, the one or more sensors may be built into the administration member 110 and/or administration member 112. In certain such embodiments, the administration member 110 and/or the administration member 112 may include a delivery needle and a wiper may be arranged along a shaft of the delivery needle. The wiper may be configured to passively or actively wick blood or other biofluids along the shaft of the delivery needle into a collector where an assay can be performed.

The one or more sensors may be physically integrated with other components of the drug delivery system 100, although they are not required to be. In some embodiments, in lieu of physical integration, the one or more sensors may have only digital integration with the remainder of the drug delivery system 100. In such embodiments, one or more of the sensors may be a standalone device that is worn by or implanted within the patient and wirelessly communicates digital information with a controller of the fluid delivery system 100 and/or an external computing device such as the patient's smartphone and/or a remote server.

In the embodiment illustrated in FIG. 1, the drug delivery system 100 includes a first sensor 116 and a second sensor 118, each being operable to sense a biological condition of the patient. The first sensor 116 may be operable to sense a level or change in a level of a biochemical and the second sensor 118 may be operable to sense the patient's core temperature and/or skin temperature, although the first and second sensors 116 and 118 are not limited to such sensing functionalities and may be operable to sense any of the biological conditions mentioned herein as well as others. In the present embodiment, the biochemical sensed by the first sensor 116 may include a cytokine and/or a biomarker indicative of CRS. In alternative embodiments, either of the first sensor 116 or the second sensor 118 may be omitted.

The drug delivery system 100 may additionally include a controller 120 configured to control the operation of various component(s) of the drug delivery system 100, including the fluid delivery system 114 and an output unit 122. Further, the controller 120 may be configured to receive and/or process information, data, signals (analog and/or digital), or other output from the first sensor 116, the second sensor 118, and/or other components of the fluid delivery system 100 or external components such as the external computing device 104. Furthermore, the controller 120 may be responsive to the output it receives from such component(s), and may be configured to automatically control the operation of certain component(s) such as the fluid delivery system 114 and/or an output unit 122 according to the programming or other configuration of the controller 120.

The controller 120 may include and/or implement its operations via an electrical device (e.g., a hardwired circuit, a microprocessor, etc.), a combination of electrical devices, a mechanical device, a combination of mechanical devices, a chemical device, a combination of chemical devices, or any combination thereof (e.g., an electromechanical device, an electrochemical device, etc.). According to those embodiments wherein the controller 120 includes a microprocessor or the like, the configuration of the controller 120 may correspond to the software or other programming of the controller 120. In some embodiments, the controller 120 may be pre-configured by a manufacturer and/or healthcare provider such that it cannot later be reconfigured by the patient or other end user; whereas, in other embodiments, the controller 120 may be configurable by any individual or entity, within reason.

In some embodiments, the controller 120 may be provided as a computing device that includes one or more processors and one or more memories in communication with or integrated with each other. The one or more processors may include, for example, one or more of a microprocessor, micro-controller, programmable logic controller, digital signal processor, microcomputer, central processing unit, field programmable gate array, logic circuitry, analog circuitry, digital circuitry, software-based processing module, and any device that manipulates signals (analog and/or digital) based on hard coding of the circuitry and/or operational instructions, or any combination thereof. The one or more memories may include a non-transitory computer-readable storage medium configured to store data, including, for example, non-transitory computer-readable instructions constituting one or more services, programs, and/or modules and any data operated on or produced by such services, programs, and/or modules. The memory may store the data on a volatile (e.g., random access memory (RAM), etc.) and/or non-volatile memory (e.g., a hard disk), and may be a removable or non-removable memory. The one or more processors may be configured to fetch and execute the instructions stored in the one or more memories in order to perform or implement various functions of the drug delivery system 100, including, for example, operating the fluid delivery system 114 to deliver the first and/or second drug products to the patient according to a dosing regimen and/or interventional dosing regimen.

In some embodiments, the controller 120 may be communicatively coupled (e.g., via wired or wireless connections) with one or more of the external computing device 104, the fluid delivery system 114, the first sensor 116, the second sensor 118, and the output unit 122 such that the controller 120 can transmit communications to and/or receive communications from one or more of the external computing device 104, the fluid delivery system 114, the first sensor 116, the second sensor 118, and the output unit 122. Such communications may be electrical and/or mechanical in nature, and/or may include information, data, and/or signals (analog and/or digital).

According to some embodiments, the controller 120 may operate the fluid delivery system 114 to deliver the first drug product stored in the first reservoir 106 to the patient in accordance with a dosing regimen for which the controller 120 has been configured. Over the course of this dosing regimen, the controller 120 may be configured to operate the fluid delivery system 114 to suspend, terminate, or throttle (e.g., reduce or inhibit) delivery of the first drug product to the patient in response to a determination that: (i) the biological condition sensed by the first sensor 116 is within or outside of a first predetermined range of values or is greater or than a first predetermined value; and/or (ii) the biological condition sensed by the second sensor 118 is within or outside of a second predetermined range of values or is greater or less than a second predetermined value. Additionally or alternatively, as part of an interventional dosing regimen, the controller 120 may be configured to operate the fluid delivery system 114 to initiate delivery of the second drug product stored in the second reservoir 108 to the patient in response to a determination that (i) and/or (ii) is satisfied. Still further, the controller may be configured to operate the output unit 122 to notify the patient and/or a healthcare provider in response to a determination that (i) and/or (ii) is satisfied. As used herein a “predetermined range of values” encompasses a fixed range of values, as well as values generated by a formula or algorithm according to one or more variables or inputs, which can be determined by, for example, patient disease state, such as baseline disease burden, prior to the infusion of the first drug product.

It should be noted that while the controller 120 may be configured to analyze the output (e.g., signals, data, information, etc.) received from the first sensor 110 and/or second sensor 112 and based on this analysis make a determination as to whether (i) and/or (ii) is satisfied, it is not required for the controller 120 to be responsible for this analysis and determination. For instance, an external computing device may be responsible for analyzing the output from the first sensor 110 and/or the second sensor 112 and then may communicate its determination with regard to (i) and/or (ii) to the controller 120.

The output unit 122 may be any device suitable for conveying information to the patient or user including a display (e.g., a liquid crystal display), a touchscreen, a light (e.g., a light emitting diode), a vibrator (e.g., an electro-mechanical vibrating element), a mechanical or color-changing flag member, a speaker, an alarm, and/or any other suitable device.

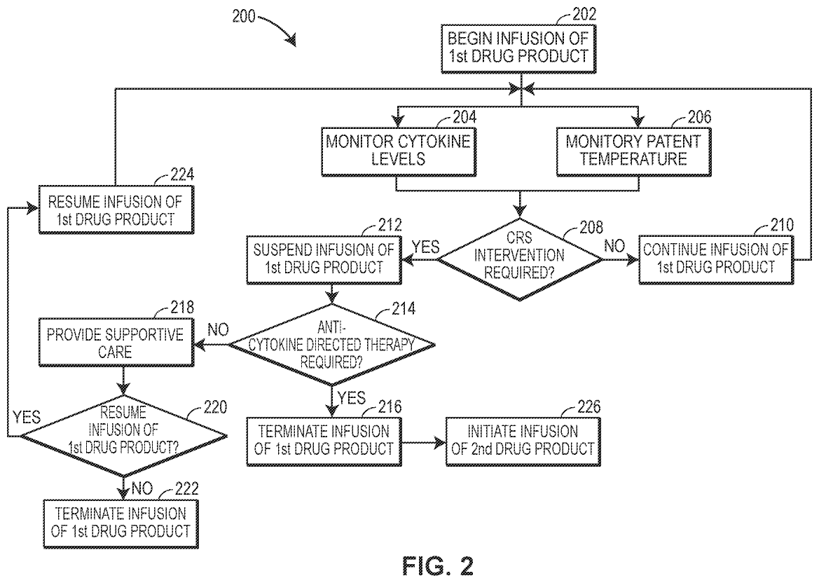

FIG. 2 illustrates a method 200 of operating a drug delivery system, such as the drug delivery system 100 in FIG. 1, to sense various biological conditions of the patient 102 and to automatically control the drug delivery system 100 according to those sense biological condition(s) such that an interventional dosing regimen can be implemented with minimal or no input from a healthcare provider. From a brief review of the flowchart of FIG. 2, it will be recognized that the method 200 according to FIG. 2 illustrates the determination of various biological conditions of the patient 102 and actions taken in response to or in association with these conditions. It should also be recognized that while the method 200 includes certain determinations and actions, other embodiments of a method of operating a drug delivery system according to the present disclosure may include only some of the determinations and actions described in connection with FIG. 2 and/or include additional determinations and actions. Further, it should be recognized that while the method 200 pertains to CRS intervention involving supportive care and/or infusion of an anti-cytokine agent, general principles associated with this method are applicable to a wide range of interventional dosing techniques in a variety of contexts.

The method 200 may start at block 202 with infusion of a first drug product including an immunotherapy agent. In some embodiments, the immunotherapy agent may include a bispecific T cell engaging (e.g., BiTE®) antibody constructs (e.g., blinatumomab) or a CAR-T cell receptor (e.g., an anti-CD19 CAR-T cell). The first drug product may be delivered to the patient 102 by automatically operating, via the controller 120, the fluid delivery system 114 to expel the first drug product form the first reservoir 106 to the patient 102 via the first administration member 110. Prior to drug delivery, the first administration member 110 may be inserted into the patient 102 so that it is in fluid communication with, for example, a vein or bodily lumen, subcutaneous tissue, etc. In some embodiments, the fluid delivery system 100 may include an insertion mechanism for automatically, upon initiation by the patient or healthcare provider, inserting the first administration member 110 at the injection site. In certain such embodiments, as a preliminary step, a housing of the insertion mechanism or the entire drug delivery system may be adhered the patient's skin. In other embodiments, the first administration member 110 may be manually inserted at the injection site.

The controller 120 may be configured (e.g., preconfigured) by the patient 102, a healthcare provider, or a device manufacturer to control the fluid delivery system 114 to infuse the first drug product to the patient 102 continuously at a specified rate and/or over a specified period of time in accordance with a prescribed dosing regimen. In some embodiments, this may involve infusing the first drug product at approximately (e.g., ±10%) 10 mL per hour for a duration of approximately (e.g., ±10%) 24 hours, or approximately (e.g., ±10%) 5 mL per hour for a duration of approximately (e.g., ±10%) 48 hours, or approximately (e.g., ±10%) 0.6 mL per hour for a duration of 7 days, or any other suitable rate and/or duration of time. According to some embodiments, a particular delivery rate may not be specified and the controller 120 may only be set with a particular time period over which drug delivery is to occur, including, for example, a duration of several minutes, several tens of minutes, an hour, hours (e.g., 2, 3, 4, 5, 6, 7, 8, 9, 10, 11, 12, 18, 24, 36, or 48 hours, or fractions thereof, such as 22.5 hours). In still further embodiments, neither a particular rate nor a particular duration of time may be configured into the controller 120, and instead the controller 120 may simply activate or release an energy source (e.g., a spring or source of pressurized gas or fluid) that naturally expels the first drug product from the first reservoir 106 over a general time frame that can vary depending on environmental factors such as backpressure, temperature, drug viscosity, etc. In embodiments where the fluid delivery system 100 is not portable and/or the duration of drug delivery is very long, the patient may have the ability, via the controller 120 or otherwise, to interrupt delivery of the first drug product so that the patient can take breaks for necessities such as eating, sleeping, etc.

Simultaneous with or shortly before or after the start of infusion of the first drug product, one or more sensors may be arranged to sense one or more biological conditions of the patient 102. The biological condition(s) may be sensed continuously or intermittently over the entire duration of method 200. Depending on the sensor, the sensor may be disposed in contact with and releasably attached to (e.g., adhered to) the surface of the patient's skin, inserted into the patient (e.g., inserted into the patient's subcutaneous tissue, inserted into an alimentary canal, etc.), implanted within the patient, or disposed within a short distance of the patient. In the present embodiment, the first sensor 116 may be inserted into the patient's tissue to sense a level or change in level of cytokines within the patient 102 (see block 204 of FIG. 2), and the second sensor 118 may be disposed in contact with the surface of the patient's skin to sense the patient's body temperature (see block 206 of FIG. 2). In embodiments where the first sensor 116 is incorporated into the first administration member 110, inserting the first sensor 116 into the patient may be accomplished by inserting the first administration member 110 into the patient. In alternative embodiments, the first sensor 116 may not be inserted within the patient but rather disposed at the surface of the patient's skin to collect sweat (e.g., eccrine sweat) and sense a level or change in level of cytokines in the collected sweat. Further, the first sensor 116 or the second sensor 118 may be omitted in certain embodiments. Still further, in some embodiments, the first sensor 116 and the second sensor 118 may be physically integrated with each other in a single unit.

Subsequently, the method 200 continues to block 208, where a determination is made whether the patient 102 is exhibiting symptoms or signs of CRS. The determination may be based on whether: (i) the biological condition sensed by the first sensor 116 is within or outside of a first predetermined range of values or is greater or than a first predetermined value; and/or (ii) the biological condition sensed by the second sensor 118 is within or outside of a second predetermined range of values or is greater or less than a second predetermined value. In the previous sentence and in other places throughout the present disclosure, the use of “and” in the “and/or” connotes that both (i) and (ii) need to be satisfied in order for there to be a determination that the patient is exhibiting signs of CRS; whereas the “or” in the “and/or” connotes that only one of (i) or (ii) needs to be satisfied in order for there to be a determination that the patient is exhibiting signs of CRS. Additional and/or alternative conditions or factors may be evaluated to determine whether patient is exhibiting signs of CRS. Furthermore, in some embodiments, the determination at block 208 may be accomplished by referencing a database, reference table, and/or algorithm, which may be stored in a memory of the controller 120 or elsewhere.

In some embodiments, the determination at block 208 may be accomplished by receiving at the controller 120 output (e.g., information, data, signals, etc.) from the first sensor 116 and/or second sensor 118, and then analyzing that output with the controller 120 to determine whether (i) and/or (ii) is satisfied. However, in alternative embodiments, the controller 120 may not be involved with this determination. Instead, for example, an external computing unit (e.g., the external computing unit 104) may receive the output from the first sensor 116 and/or second sensor 118 (e.g., via wired or wireless communications, directly or indirectly received from the sensor(s)) and then analyze the output to determine whether (i) and/or (ii) is satisfied. The external computing unit thereafter may notify the controller 120 of its determination (e.g., via a wired or wireless communications) and the controller 120 may take appropriate action based on the determination.

If CRS intervention is not determined as being necessary at block 208, the method 200 may continue with infusion of the first drug product and continue with monitoring the one or more biological conditions of the patient 102 (see block 210). On the other hand, if CRS intervention is determined to be necessary or recommended at block 208, the method 200 may proceed to block 212. At block 212, the controller 120, according to its programming or other configuration, may operate the fluid delivery system 114 to suspend or throttle delivery of the first drug product to the patient. In some embodiments, the controller 120 may also at this stage control the output unit 122 to notify (e.g., via visual and/or audio output) the patient and/or a healthcare provider that the symptoms of CRS have been detected and/or that the administration of the first drug product is consequently being suspended or throttled. Additionally or alternatively, the controller 120 may transmit a signal to the external computing device 104, which may function as an output unit, such that a remote healthcare provider, family member, friend, and/or other individual or entity can be notified via the external computing device 104 that the patient 102 is experiencing symptoms of CRS.

As an alternative to having the controller 120 automatically suspend or throttle delivery of the first drug product at block 212, delivery of the first drug product can be manually interrupted by the patient 102 and/or a healthcare provider after receiving a notification about the onset of CRS via the output unit 122, the external computing device 104, and/or another device.

After or simultaneous with the action at block 212, a determination may be made as to whether administration of an anti-cytokine agent is needed in order to treat the symptoms of CRS (see block 214 in FIG. 2). This determination may be made by the controller 120, or another device such as the external computing unit 104. Further, this determination may be based on the degree or extent to which: (i) the biological condition sensed by the first sensor 116 is within or outside of the first predetermined range of values or is greater or than the first predetermined value; and/or (ii) the biological condition sensed by the second sensor 118 is within or outside of the second predetermined range of values or is greater or less than the second predetermined value. Additional and/or alternative conditions or factors may also be evaluated at block 214.

If administration of the anti-cytokine agent is determined to be unnecessary at block 214, the method 200 may proceed to block 218. Here, a healthcare provider and/or the patient may be instructed to provide supportive care that does not involve administration of an anti-cytokine agent in an effort to mitigate the effects of CRS. This instruction, which may be generated by the controller 120, may be communicated to the healthcare provider and/or patient via the output unit 122 and/or the external computing device 104. The instruction may be visual, audio, and/or any other form of communication. In some embodiments, the supportive care may include administering, intravenously or otherwise, an antipyretic drug product and/or an IV fluid to the patient 102. This administration step may be performed manually in some embodiments and/or may not involve the drug delivery system 100. However, in other embodiments, the fluid delivery system 100 may include one or more reservoirs containing the antipyretic drug product and/or IV fluid, and the controller 120 may automatically operate the fluid delivery system 114 to deliver the antipyretic drug product and/or IV fluid to the patient via an administration member (including, but not limited to, the first administration member 110, the second administration member 112, or another administration member).

While supportive care is being provided at block 218, the one or more sensors may continue to monitor the one or more biological conditions of the patient. Subsequently, after a predetermined time period, for example, a determination made be made as to whether infusion of the first drug product can be resumed in view of the fact that the symptoms of CRS have subsided (see block 220). This determination may be made by the controller 120, or another device such as the external computing unit 104. Further, this determination may similar to the determination performed at block 208, except the reverse. Additional and/or alternative conditions or factors may be evaluated at block 218.

If resuming delivery of the first drug product is determined to be appropriate at block 218, the controller 120 may automatically control the fluid delivery system 114 to resume delivery of the first drug product from the first reservoir 106 to the patient 102. Thereafter, the method 200 may start over again. By contrast, if the determination at block 218 is that resuming delivery of the first drug product is not appropriate, then the controller 120 may terminate or prevent any additional delivery of the first drug product to the patient 102 and the method 200 may come to an end.

Referring back to block 214, if administration of the anti-cytokine agent is determined to be required at block 214, the method 200 may proceed to block 216. Here, the controller 120 may terminate any additional delivery of the first drug product to the patient 102. In alternative embodiments, the controller 120 may only suspend or throttle delivery of the first drug product to the patient at block 216.

Next, the method 200 may proceed to block 226, where delivery of the second drug product from the second reservoir 108 to the patient 102 may be initiated. The second drug product may treat CRS, or another condition or syndrome induced by administration of the first drug product. In some embodiments, the second drug product may include an anti-cytokine agent. The anti-cytokine agent may include, for example, a corticosteroid (e.g., dexamethasone), an anti-interleukin-6 (IL-6) receptor antibody (e.g., tocilizumab), and/or an anti-IL-6 chimeric monoclonal antibody (e.g., siltuximab). The second drug product may be delivered to the patient 102 by automatically operating, via the controller 120, the fluid delivery system 114 to expel the second drug product form the second reservoir 108 to the patient 102 via the second administration member 112. Prior to the start of the method 200 or immediately prior to block 226, the second administration member 112 may be inserted into the patient 102 so that it is in fluid communication with, for example, a vein or bodily lumen, subcutaneous tissue, etc. In some embodiments, the fluid delivery system 100 may include may include an insertion mechanism for automatically, upon initiation by the patient or healthcare provider, inserting the second administration member 112 at the injection site. In certain such embodiments, as a preliminary step, a housing of the insertion mechanism or the entire drug delivery system may be adhered to the patient's skin. In other embodiments, the second administration member 112 may be manually inserted in a vein or at the injection site.

In still further alternative embodiments, the second drug product may be delivered to the patient 102 via the first administration member 110. In such alternative embodiments, the controller 120 may control a valve member such that the first administration member 110 is in fluid communication with the second reservoir 108 instead of the first reservoir 106 prior to delivery of the second drug product.

The controller 120 may be configured (e.g., preconfigured) by the patient 102, a healthcare provider, or a device manufacturer to control the fluid delivery system 114 to infuse the second drug product to the patient 102 continuously at a specified rate and/or over a specified period of time in accordance with a prescribed dosing regimen. In some embodiments, this may involve infusing the second drug product at approximately (e.g., ±10%) 10 mL per hour for a duration of approximately (e.g., ±10%) 2 hours, or approximately (e.g., ±10%) 5 mL per hour for a duration of approximately (e.g., ±10%) 4 hours, or any other suitable rate and/or duration of time. According to some embodiments, a particular delivery rate may not be specified and the controller 120 may only be set with a particular time period over which delivery of the second drug product is to occur, including, for example, a duration of several minutes, several tens of minutes, an hour, hours (e.g., 2, 3, 4, 5, 6, 7, 8, 9, 10, 11, 12, 18, 24, 36, or 48 hours, or fractions thereof, such as 22.5 hours). In still further embodiments, neither a particular rate or duration may be configured into the controller 120, and instead the controller 120 may simply activate or release an energy source (e.g., a spring or source of pressurized gas or fluid) that naturally expels the second drug product from the second reservoir 108 over a general time frame that can vary depending on environmental factors such as backpressure, temperature, drug viscosity, etc. In embodiments where the fluid delivery system 100 is not portable and/or the duration of delivery of the second drug product is very long, the patient may have the ability, via the controller 120 or otherwise, to interrupt delivery of the second drug product so that the patient can take breaks for necessities such as eating, sleeping, etc.

The foregoing method advantageously provides for the automatic detection and treatment of CRS or other syndrome or condition induced by administration of a drug product. This may lead to earlier identification of CRS or other syndrome or condition, thereby increasing the likelihood that it can be treated successfully. Furthermore, the burden on healthcare providers to monitor a patient for side effects such as CRS following a drug treatment is alleviated.

It should be recognized that according to other embodiments of the disclosure, various aspects of the method 200 described in connection to FIG. 2 may be considered to be optional or omitted. For example, it may not be necessary to make the determination at block 214 relative to whether an anti-cytokine agent should be administered. If the determination at block 214 is omitted, the method proceeds directly from block 208 to block 216 in the event that CRS intervention is determined to be needed at block 208.

Referring now to FIG. 3, described is an on-body injector implementation of the foregoing drug delivery system 100. Various elements of the on-body injector illustrated in FIG. 3 are similar in function to elements of the drug delivery system 100 illustrated in FIG. 1. Those elements are assigned with the same references numerals in FIG. 3 as in FIG. 1, except they are incremented by 200 in FIG. 3. A description of some of these elements is abbreviated or eliminated in the interest of conciseness. Moreover, the on-body injector illustrated in FIG. 3 may be used in accordance with the method 200 described in connection with FIG. 2.

FIG. 3 illustrates an on-body injector 300 including an insertion mechanism 305, a first reservoir 306, a second reservoir 308, a fluid pathway connection assembly 307, a fluid delivery system 314, and a controller 320, each of which may be disposed within an interior space of a main housing 329. An actuator 328 (e.g., a user-depressible button, touchscreen, microphone, etc.) may protrude through or otherwise be disposed at an exterior surface of the housing 329 and may be configured to initiate operation of the on-body injector 300 by activating, via mechanical and/or electrical means (shown in dotted lines in FIG. 3), the insertion mechanism 305, the fluid pathway connection assembly 307, the fluid delivery system 314, the controller 320, and/or other mechanisms and/or electronics. In embodiments where the actuator 328 is a button that is depressed or otherwise physically moved by a user or patient, the actuator 328 may be configured to exert a motive force needed to activate the insertion mechanism 305, the fluid pathway connection assembly 307, the fluid delivery system 314, the controller 320, and/or other elements. In such embodiments, the actuator 328 may be physically connected to, either directly or indirectly via a mechanical linkage, the insertion mechanism 305, the fluid delivery system 314, the fluid pathway connection assembly 307, and/or other mechanisms such that manually depressing or otherwise interacting with the actuator 328 supplies the motive force necessary to activate the insertion mechanism 305, the fluid pathway connection assembly 307, the fluid delivery system 314, and/or other elements. For example, in some embodiments, manually depressing the actuator 328 may cause the fluid pathway connection assembly 307 to move towards the stationarily-positioned reservoirs 306 and 308, or alternatively, cause the movable reservoirs 306 and 308 to move towards the stationarily-positioned fluid pathway connection assembly 307, and thereby cause container access needles to penetrate through respective seal members into respective drug-containing chambers of the reservoirs 306 and 308. Additionally or alternatively, the actuator 328 may operate as an input device that transmits an electrical and/or mechanical signal to the controller 320, which in turn may execute programmable instructions to control operation of the insertion mechanism 305, the fluid delivery system 314, the fluid pathway connection assembly 305, and/or other elements. In such embodiments, the controller 320 may include a processor (e.g., a microprocessor) and a non-transitory memory for storing the programmable instructions to be executed by the processor. Furthermore, in such embodiments, the fluid delivery system 314 may include an internal actuator (e.g., an electric motor, a pneumatic or hydraulic pump, and/or a source of pressurized gas or liquid) which is separate from the actuator 328 and which, in response to an electrical control signal received from the controller 320, exerts the motive force needed to activate the insertion mechanism 305, the fluid pathway connection assembly 307, the fluid delivery system 314, and/or other elements.

With continued reference to FIG. 3, the housing 329 may include a bottom wall 325 configured to be releasably attached (e.g., adhered with an adhesive) to skin 311 of the patient, and a top wall 327 including an output unit 322 (e.g., visual and/or audio indicators such as lights, a graphical display(s), speaker, etc.) and/or a window 335 for viewing the reservoirs 306 and 308. An opening 331 may be formed in the bottom wall 325, and optionally a pierceable sterile barrier 333, such as a pierceable septum, may extend across the opening 331 to seal the interior of the housing 329 prior to use. In some embodiments, the pierceable sterile barrier 333 may be omitted, and instead a removable sealing member (not illustrated) may cover and seal close the opening 331 prior to use.

After the bottom wall 325 of the housing 329 is attached to the patient's skin 311, the insertion mechanism 305 may be activated to move an administration member 310, here including a cannula 323, from a retracted position within the housing 329 to a deployed position extending outside of the housing 329. In the present embodiment, this may include the insertion mechanism 305 inserting a trocar or introducer member 321 and the cannula 323 surrounding the introducer member 321 through the pierceable sterile barrier 333 and into the patient's tissue, as illustrated in FIG. 3. Immediately or shortly thereafter, the insertion mechanism 305 may automatically retract the introducer member 321, leaving the distal open end of the cannula 323 inside the patient for subcutaneous delivery of the first drug product and the second drug product from, respectively, the reservoirs 306 and 308. The introducer member 321 may be solid and have a sharpened end for piercing the patient's skin 311. Furthermore, the introducer member 321 may be made of a material that is more rigid than the cannula 323. In some embodiments, the introducer member 321 may be made of metal, whereas the cannula 323 may be made of plastic or another polymer. The relative flexibility of the cannula 323 may allow it to be disposed subcutaneously within the patient's tissue for a period of a time without causing pain or significant discomfort to the patient. In other embodiments (not illustrated), the introducer member 321 and cannula 323 may be omitted, and instead the insertion mechanism 305 may insert only a rigid, hollow needle into the patient for subcutaneous delivery of the drug products.

In some embodiments, the insertion mechanism 305 may include one or more springs (e.g., helical compression springs, a helical extension springs, a helical torsion springs, a spiral torsion springs, etc.) initially retained in an energized state, and which are released upon depression of the actuator 328 in order to insert the introducer member 321 and cannula 323, or a rigid hollow needle, into the patient. Furthermore, retraction of the introducer member 321 may be achieved by the automatic release of another spring after the introducer member 321 and cannula 323 have been inserted into the patient. Other power sources for insertion and/or retraction are possible, including, for example, an electric motor, a hydraulic or pneumatic pump, or a canister that releases a pressurized gas or pressurized liquid to provide actuation energy.

Still referring to FIG. 3, the first reservoir 306, which in some contexts may be referred to as a primary container, may include a wall 338a with an interior surface defining an interior space that is filled or fillable with the first drug product, and an exterior surface. In some embodiments, the first reservoir 306 may be pre-filled with the first drug product by a drug manufacturer prior to installation of the first reservoir 306 in the on-body injector 300. In some embodiments, the first reservoir 306 may be rigidly connected to the housing 329 such that the first reservoir 306 cannot move relative to the housing; whereas, in other embodiments, the first reservoir 306 may be slidably connected to the housing 329 such that the first reservoir 306 can move relative to the housing 329 during operation of the on-body injector 300. The first reservoir 306 may have an elongate, barrel-like or cylindrical shape extending along a longitudinal axis A1. In some embodiments, the longitudinal axis A1 of the first reservoir 306 may be perpendicular or substantially perpendicular, or otherwise non-parallel, to a direction in which the insertion mechanism 305 inserts the administration member 310 into the patient. This configuration may allow the on-body injector to have a generally planar, low-profile shape that can be worn by the patient without impeding the patient's movement. Initially, a stopper 334a or other plunger member may be positioned in the first reservoir 306 at a proximal end 336a of the first reservoir 306. The stopper 334a may sealingly and slidably engage the interior surface of the wall 338a of the first reservoir 306, and may be movable relative to the wall 338a of the first reservoir 306.

The second reservoir 308 may be configured in a similar manner as the first reservoir 306. Similar components are denoted with the suffix “b” instead of the suffix “a” in FIG. 3 relative to the second reservoir 308.

While the first and second reservoirs 306 and 308 in the illustrated embodiment are stacked vertically on top of each other, in alternative embodiments the first and second reservoirs 306 and 308 may be arranged on a common horizontal plane, so as to limit the height of the on-body injector.

The volume of the first drug product contained in the first reservoir 306 or the volume of the second drug product container in the second reservoir 308 may be: any volume in a range between approximately (e.g., ±10%) 0.5-100 mL, or any volume in a range between approximately (e.g., ±10%) 0.5-50 mL, or any volume in a range between approximately (e.g., ±10%) 0.5-25 mL, any volume in a range between approximately (e.g., ±10%) 0.5-10 mL, or any volume in a range between approximately (e.g., ±10%) 1-10 mL, or any volume in a range between approximately (e.g., ±10%) 1-8 mL, or any volume in a range between approximately (e.g., ±10%) 1-5 mL, or any volume in a range between approximately (e.g., ±10%) 1-3 mL, or any volume equal to or greater than approximately (e.g., ±10%) 3 mL, or any volume equal to or greater than approximately (e.g., ±10%) 10 mL, or any volume equal to or greater than approximately (e.g., ±10%) 25 mL, or any volume equal to or greater than approximately (e.g., ±10%) 50 mL, or any volume equal to or greater than approximately (e.g., ±10%) 60 mL, or any volume equal to or greater than approximately (e.g., ±10%) 75 mL.

During operation of the on-body injector 300, the fluid delivery system 314 may selectively push the stoppers 334a and 334b along their respective longitudinal axes A1 and A2 from the proximal end 336a or 336b to the distal end 337a or 337b of their respective reservoir in order to expel the first and second drug products from their respective reservoirs one at a time. In alternative embodiments, the fluid delivery system 314 may be configured to push the stoppers 334a and 334b simultaneously to expel the first and second drug products simultaneously. In some embodiments, the fluid delivery system 314 may include one or more springs (e.g., helical compression springs, a helical extension springs, a helical torsion springs, a spiral torsion springs, etc.) initially retained in an energized state, and which are released upon depression of the actuator 328 and/or another actuator. Following their release, the spring(s) may expand or contract to move the stoppers 334a and 334b through their respective reservoirs to expel the drug products contained therein. In other embodiments, the fluid delivery system 314 may include an electric motor which rotates a gear mechanism, including for example one or more sprocket gears, to cause axial motion of the stoppers 334a and 336b through their respective reservoirs. In still further embodiments, the fluid delivery system 314 may include both an electric motor and spring(s), wherein the electric motor regulates expansion of the spring(s) via a tether or pulley system. In still further embodiments, the fluid delivery system 314 may include a canister that releases a pressurized gas or pressurized liquid to provide actuation energy.

At the distal ends 337a or 337b of each reservoir, an opening may be formed in a distal end surface of the wall 338a or 338b. The distal end surface may define a portion of the exterior surface or of the wall 338a or 338b. Prior to operation of the on-body injector 300, the opening may be covered and sealed closed by a seal member 340a or 340b, such as a pierceable septum, connected to the distal ends 337a or 337b of the respective reservoirs. Generally, the seal members 340a and 340b may be configured to selectively permit access to, respectively, the reservoirs 306 and 308. During operation, the seal members 340a and 340b may be physically altered (e.g., pierced) to permit fluid communication with the first and second drug products in the reservoirs 306 and 308. In some embodiments, the seal members 340a and 340b may be constructed of a flexible or elastically deformable material such as rubber, for example, which is capable of being penetrated or pierced by, respectively, a sharpened end or point 363a or 363b of a container access needle 360a or 360b of the fluid pathway connection assembly 307.

Still referring to FIG. 3, the fluid pathway connection assembly 307 may be configured to selectively establish fluid communication between each of the reservoirs 306 or 308 and the insertion mechanism 305 via a sterile fluid flow path during use of the on-body injector 300. Prior to use of the on-body injector 300, the fluid pathway connection assembly 307 may not be in fluid communication with either of the reservoirs 306 and 308. During setup of the on-body injector 300, or during operation of the on-body injector 300 but prior to drug delivery, the user may manually, or the on-body injector 300 may automatically, enable, connect, or open the necessary connections to establish fluid communication between the fluid pathway connection assembly 307, on the one hand, and the first reservoir 306 and/or the second reservoir 308. Subsequently, the fluid delivery system 314 may selectively move the stoppers 334a and 334b in the distal direction to selectively force the first and second drug products through the sterile fluid flow path of the fluid pathway connection assembly 307 and into the cannula 333 or needle or other administration member for subcutaneous delivery to the patient.

The fluid pathway connection assembly 307 may include a first end 344 selectively connected in fluid communication to the first and second reservoirs 306 and 308, a second end 348 connected in fluid communication with the insertion mechanism 305, and a fluid passage 350 providing fluid communication between the first end 344 and the second end 348. The fluid passage 350 may be sterilized, and may be partially or entirely made of a flexible tubing 352. Initially, there may be slack in the flexible tubing 352 to allow the fluid pathway connection assembly 307 to move relative to the housing 329 and/or to allow components of the insertion mechanism 305 to which the fluid pathway connection assembly 307 is attached to move relative to the housing 329.

Still referring to FIG. 3, the first end 344 of the fluid pathway connection assembly 307 may include first and second container access needles 360a and 360b. Prior to activation of the fluid pathway connection assembly 307, the container access needle 360a and 360b may be retained in a storage position wherein the proximal ends of the container access needles 360a and 360b each is disposed exterior to, and thus not in fluid communication with, respectively, the first and second reservoirs 306 and 308 (as seen in FIG. 3). During operation of the fluid pathway connection assembly 307, the first container access needle 360a may move toward the first reservoir 306 and into an operational position wherein the proximal end of the first container access needle 360a is in fluid communication with the first reservoir 306. Simultaneously with this action, or at a later time during use of the on-body injector 300, the fluid pathway connection assembly 307 may move the second container access needle 360b toward the second reservoir 308 and into an operational position wherein the proximal end of the second container access needle 360b is in fluid communication with second reservoir 308. Subsequently, the fluid delivery system 314 may move the stopper 334a and/or the stopper 334b in the distal direction to expel the first drug product and/or the second drug product stored in the reservoirs through the respective container access needle 360a and 360b, then through a sterile fluid flow path of the fluid pathway connection assembly 307, and then into the cannula 323 or needle or other administration member of the insertion mechanism 305 for subcutaneous delivery to the patient.

In some embodiments, the fluid pathway connection assembly 307 may include a valve member 380 that is actuatable by the controller 320 to selectively permit fluid communication between the first container access needle 360a and the fluid passage 350, or alternatively, between the second container access needle 360b and the fluid passage 350.

The on-body injector 300 may also include one or more sensors operable to sense one or more biological conditions of the patient. The one or more sensors can be any of those described above in connection with the drug delivery system 100 or other types of sensors. In the embodiment illustrated in FIG. 3, a first sensor 316 and a second sensor 318 are arranged at the bottom wall 325 of the housing 329. The first sensor 316 comprises an array of microneedles insertable into the patient's skin 311, and the second sensor 318 includes a thermocouple or a biochemical sensor (e.g., a skin or sweat sensor) arranged to contact but not penetrate the surface of the patient's skin 311. Other configurations of the first and second sensors 316 and 318 are also possible. The first sensor 316 may be operable to sense a level or change in a level of a biochemical, and the second sensor 318 may be operable to sense the patient's core temperature, skin temperature, and/or a level or change in level of a biochemical, although the first and second sensors 316 and 318 are not limited to such sensing functionalities and may be operable to sense any of the biological conditions mentioned herein as well as others. In the present embodiment, the biochemical sensed by the first sensor 316 may include a cytokine, chemokine, and/or other biomarker indicative of CRS. In alternative embodiments, either of the first sensor 116 or the second sensor 118 may be omitted. Additionally sensors may also be included depending on the biological conditions to be monitored.

Except where differences in structure or configuration require otherwise, the on-body injector 300 illustrated in FIG. 3 may operate in a similar manner as the drug delivery system 100 described above and/or may be used to implement the interventional dosing regimen described in connection with the method 200 of FIG. 2 or certain portions thereof.

As will be recognized, the systems, devices, and methods according to the present disclosure may have one or more advantages relative to conventional technology, any one or more of which may be present in a particular embodiment in accordance with the features of the present disclosure included in that embodiment. Other advantages not specifically listed herein may also be recognized as well.

Drug Information

The above description describes various assemblies, devices, and methods for use with a drug delivery system or device. It should be clear that the assemblies, drug delivery systems or devices, or methods can further comprise use of a medicament listed below with the caveat that the following list should neither be considered to be all inclusive nor limiting. The medicament will be contained in a reservoir. In some instances, the reservoir is a primary container that is either filled or pre-filled for treatment with the medicament. The primary container can be a cartridge or a pre-filled syringe, or a non-rigid collapsible pouch, such as an IV bag.

For example, the drug delivery device or more specifically the reservoir of the device may be filled with colony stimulating factors, such as granulocyte colony-stimulating factor (G-CSF). Such G-CSF agents include, but are not limited to, Neupogen® (filgrastim) and Neulasta® (pegfilgrastim). In various other embodiments, the drug delivery device may be used with various pharmaceutical products, such as an erythropoiesis stimulating agent (ESA), which may be in a liquid or a lyophilized form. An ESA is any molecule that stimulates erythropoiesis, such as Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin zeta, epoetin theta, and epoetin delta, as well as the molecules or variants or analogs thereof as disclosed in the following patents or patent applications, each of which is herein incorporated by reference in its entirety: U.S. Pat. Nos. 4,703,008; 5,441,868; 5,547,933; 5,618,698; 5,621,080; 5,756,349; 5,767,078; 5,773,569; 5,955,422; 5,986,047; 6,583,272; 7,084,245; and 7,271,689; and PCT Publication Nos. WO 91/05867; WO 95/05465; WO 96/40772; WO 00/24893; WO 01/81405; and WO 2007/136752.

An ESA can be an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, epoetin alfa, epoetin beta, epoetin delta, epoetin omega, epoetin iota, epoetin zeta, and analogs thereof, pegylated erythropoietin, carbamylated erythropoietin, mimetic peptides (including EMP1/hematide), and mimetic antibodies. Exemplary erythropoiesis stimulating proteins include erythropoietin, darbepoetin, erythropoietin agonist variants, and peptides or antibodies that bind and activate erythropoietin receptor (and include compounds reported in U.S. Publication Nos. 2003/0215444 and 2006/0040858, the disclosures of each of which is incorporated herein by reference in its entirety) as well as erythropoietin molecules or variants or analogs thereof as disclosed in the following patents or patent applications, which are each herein incorporated by reference in its entirety: U.S. Pat. Nos. 4,703,008; 5,441,868; 5,547,933; 5,618,698; 5,621,080; 5,756,349; 5,767,078; 5,773,569; 5,955,422; 5,830,851; 5,856,298; 5,986,047; 6,030,086; 6,310,078; 6,391,633; 6,583,272; 6,586,398; 6,900,292; 6,750,369; 7,030,226; 7,084,245; and 7,217,689; U.S. Publication Nos. 2002/0155998; 2003/0077753; 2003/0082749; 2003/0143202; 2004/0009902; 2004/0071694; 2004/0091961; 2004/0143857; 2004/0157293; 2004/0175379; 2004/0175824; 2004/0229318; 2004/0248815; 2004/0266690; 2005/0019914; 2005/0026834; 2005/0096461; 2005/0107297; 2005/0107591; 2005/0124045; 2005/0124564; 2005/0137329; 2005/0142642; 2005/0143292; 2005/0153879; 2005/0158822; 2005/0158832; 2005/0170457; 2005/0181359; 2005/0181482; 2005/0192211; 2005/0202538; 2005/0227289; 2005/0244409; 2006/0088906; and 2006/0111279; and PCT Publication Nos. WO 91/05867; WO 95/05465; WO 99/66054; WO 00/24893; WO 01/81405; WO 00/61637; WO 01/36489; WO 02/014356; WO 02/19963; WO 02/20034; WO 02/49673; WO 02/085940; WO 03/029291; WO 2003/055526; WO 2003/084477; WO 2003/094858; WO 2004/002417; WO 2004/002424; WO 2004/009627; WO 2004/024761; WO 2004/033651; WO 2004/035603; WO 2004/043382; WO 2004/101600; WO 2004/101606; WO 2004/101611; WO 2004/106373; WO 2004/018667; WO 2005/001025; WO 2005/001136; WO 2005/021579; WO 2005/025606; WO 2005/032460; WO 2005/051327; WO 2005/063808; WO 2005/063809; WO 2005/070451; WO 2005/081687; WO 2005/084711; WO 2005/103076; WO 2005/100403; WO 2005/092369; WO 2006/50959; WO 2006/02646; and WO 2006/29094.

Examples of other pharmaceutical products for use with the device may include, but are not limited to, antibodies such as Vectibix® (panitumumab), Xgeva™ (denosumab) and Prolia™ (denosamab); other biological agents such as Enbrel® (etanercept, TNF-receptor/Fc fusion protein, TNF blocker), Neulasta® (pegfilgrastim, pegylated filgastrim, pegylated G-CSF, pegylated hu-Met-G-CSF), Neupogen® (filgrastim, G-CSF, hu-MetG-CSF), and Nplate® (romiplostim); small molecule drugs such as Sensipar® (cinacalcet). The device may also be used with a therapeutic antibody, a polypeptide, a protein or other chemical, such as an iron, for example, ferumoxytol, iron dextrans, ferric glyconate, and iron sucrose. The pharmaceutical product may be in liquid form, or reconstituted from lyophilized form.

Among particular illustrative proteins are the specific proteins set forth below, including fusions, fragments, analogs, variants or derivatives thereof:

OPGL specific antibodies, peptibodies, and related proteins, and the like (also referred to as RANKL specific antibodies, peptibodies and the like), including fully humanized and human OPGL specific antibodies, particularly fully humanized monoclonal antibodies, including but not limited to the antibodies described in PCT Publication No. WO 03/002713, which is incorporated herein in its entirety as to OPGL specific antibodies and antibody related proteins, particularly those having the sequences set forth therein, particularly, but not limited to, those denoted therein: 9H7; 18B2; 2D8; 2E11; 16E1; and 22B3, including the OPGL specific antibodies having either the light chain of sequence identification number 2 as set forth therein in FIG. 2 and/or the heavy chain of sequence identification number 4, as set forth therein in FIG. 4, each of which is individually and specifically incorporated by reference herein in its entirety fully as disclosed in the foregoing publication;

Myostatin binding proteins, peptibodies, and related proteins, and the like, including myostatin specific peptibodies, particularly those described in U.S. Publication No. 2004/0181033 and PCT Publication No. WO 2004/058988, which are incorporated by reference herein in their entirety particularly in parts pertinent to myostatin specific peptibodies, including but not limited to peptibodies of the mTN8-19 family, including those of sequence identification numbers 305-351, including TN8-19-1 through TN8-19-40, TN8-19 con1 and TN8-19 con2; peptibodies of the mL2 family of sequence identification numbers 357-383; the mL15 family of sequence identification numbers 384-409; the mL17 family of sequence identification numbers 410-438; the mL20 family of sequence identification numbers 439-446; the mL21 family of sequence identification numbers 447-452; the mL24 family of sequence identification numbers 453-454; and those of sequence identification numbers 615-631, each of which is individually and specifically incorporated by reference herein in their entirety fully as disclosed in the foregoing publication;

IL-4 receptor specific antibodies, peptibodies, and related proteins, and the like, particularly those that inhibit activities mediated by binding of IL-4 and/or IL-13 to the receptor, including those described in PCT Publication No. WO 2005/047331 or PCT Application No. PCT/US2004/37242 and in U.S. Publication No. 2005/112694, which are incorporated herein by reference in their entirety particularly in parts pertinent to IL-4 receptor specific antibodies, particularly such antibodies as are described therein, particularly, and without limitation, those designated therein: L1H1; L1H2; L1H3; L1H4; L1H5; L1H6; L1H7; L1H8; L1H9; L1H10; L1H11; L2H1; L2H2; L2H3; L2H4; L2H5; L2H6; L2H7; L2H8; L2H9; L2H10; L2H11; L2H12; L2H13; L2H14; L3H1; L4H1; L5H1; L6H1, each of which is individually and specifically incorporated by reference herein in its entirety fully as disclosed in the foregoing publication;

Interleukin 1-receptor 1 (“IL1-R1”) specific antibodies, peptibodies, and related proteins, and the like, including but not limited to those described in U.S. Publication No. 2004/097712, which is incorporated herein by reference in its entirety in parts pertinent to IL1-R1 specific binding proteins, monoclonal antibodies in particular, especially, without limitation, those designated therein: 15CA, 26F5, 27F2, 24E12, and 10H7, each of which is individually and specifically incorporated by reference herein in its entirety fully as disclosed in the aforementioned publication;

Ang2 specific antibodies, peptibodies, and related proteins, and the like, including but not limited to those described in PCT Publication No. WO 03/057134 and U.S. Publication No. 2003/0229023, each of which is incorporated herein by reference in its entirety particularly in parts pertinent to Ang2 specific antibodies and peptibodies and the like, especially those of sequences described therein and including but not limited to: L1(N); L1(N) WT; L1(N) 1K WT; 2xL1(N); 2xL1(N) WT; Con4 (N), Con4 (N) 1K WT, 2xCon4 (N) 1K; L1C; L1C 1K; 2xL1C; Con4C; Con4C 1K; 2xCon4C 1K; Con4-L1 (N); Con4-L1C; TN-12-9 (N); C17 (N); TN8-8(N); TN8-14 (N); Con 1 (N), also including anti-Ang 2 antibodies and formulations such as those described in PCT Publication No. WO 2003/030833 which is incorporated herein by reference in its entirety as to the same, particularly Ab526; Ab528; Ab531; Ab533; Ab535; Ab536; Ab537; Ab540; Ab543; Ab544; Ab545; Ab546; A551; Ab553; Ab555; Ab558; Ab559; Ab565; AbF1AbFD; AbFE; AbFJ; AbFK; AbG1D4; AbGC1E8; AbH1C12; AbIA1; AbIF; AbIK, AbIP; and AbIP, in their various permutations as described therein, each of which is individually and specifically incorporated by reference herein in its entirety fully as disclosed in the foregoing publication;

NGF specific antibodies, peptibodies, and related proteins, and the like including, in particular, but not limited to those described in U.S. Publication No. 2005/0074821 and U.S. Pat. No. 6,919,426, which are incorporated herein by reference in their entirety particularly as to NGF-specific antibodies and related proteins in this regard, including in particular, but not limited to, the NGF-specific antibodies therein designated 4D4, 4G6, 6H9, 7H2, 14D10 and 14D11, each of which is individually and specifically incorporated by reference herein in its entirety fully as disclosed in the foregoing publication;

CD22 specific antibodies, peptibodies, and related proteins, and the like, such as those described in U.S. Pat. No. 5,789,554, which is incorporated herein by reference in its entirety as to CD22 specific antibodies and related proteins, particularly human CD22 specific antibodies, such as but not limited to humanized and fully human antibodies, including but not limited to humanized and fully human monoclonal antibodies, particularly including but not limited to human CD22 specific IgG antibodies, such as, for instance, a dimer of a human-mouse monoclonal hLL2 gamma-chain disulfide linked to a human-mouse monoclonal hLL2 kappa-chain, including, but limited to, for example, the human CD22 specific fully humanized antibody in Epratuzumab, CAS registry number 501423-23-0;

IGF-1 receptor specific antibodies, peptibodies, and related proteins, and the like, such as those described in PCT Publication No. WO 06/069202, which is incorporated herein by reference in its entirety as to IGF-1 receptor specific antibodies and related proteins, including but not limited to the IGF-1 specific antibodies therein designated L1H1, L2H2, L3H3, L4H4, L5H5, L6H6, L7H7, L8H8, L9H9, L10H10, L11H11, L12H12, L13H13, L14H14, L15H15, L16H16, L17H17, L18H18, L19H19, L20H20, L21H21, L22H22, L23H23, L24H24, L25H25, L26H26, L27H27, L28H28, L29H29, L30H30, L31H31, L32H32, L33H33, L34H34, L35H35, L36H36, L37H37, L38H38, L39H39, L40H40, L41H41, L42H42, L43H43, L44H44, L45H45, L46H46, L47H47, L48H48, L49H49, L50H50, L51H51, L52H52, and IGF-1R-binding fragments and derivatives thereof, each of which is individually and specifically incorporated by reference herein in its entirety fully as disclosed in the foregoing publication;

Also among non-limiting examples of anti-IGF-1R antibodies for use in the methods and compositions of the present invention are each and all of those described in:

(i) U.S. Publication No. 2006/0040358 (published Feb. 23, 2006), 2005/0008642 (published Jan. 13, 2005), 2004/0228859 (published Nov. 18, 2004), including but not limited to, for instance, antibody 1A (DSMZ Deposit No. DSM ACC 2586), antibody 8 (DSMZ Deposit No. DSM ACC 2589), antibody 23 (DSMZ Deposit No. DSM ACC 2588) and antibody 18 as described therein;

(ii) PCT Publication No. WO 06/138729 (published Dec. 28, 2006) and WO 05/016970 (published Feb. 24, 2005), and Lu et al. (2004), J. Biol. Chem. 279:2856-2865, including but not limited to antibodies 2F8, A12, and IMC-A12 as described therein;

(iii) PCT Publication No. WO 07/012614 (published Feb. 1, 2007), WO 07/000328 (published Jan. 4, 2007), WO 06/013472 (published Feb. 9, 2006), WO 05/058967 (published Jun. 30, 2005), and WO 03/059951 (published Jul. 24, 2003);

(iv) U.S. Publication No. 2005/0084906 (published Apr. 21, 2005), including but not limited to antibody 7C10, chimaeric antibody C7C10, antibody h7C10, antibody 7H2M, chimaeric antibody *7C10, antibody GM 607, humanized antibody 7C10 version 1, humanized antibody 7C10 version 2, humanized antibody 7C10 version 3, and antibody 7H2HM, as described therein;

(v) U.S. Publication Nos. 2005/0249728 (published Nov. 10, 2005), 2005/0186203 (published Aug. 25, 2005), 2004/0265307 (published Dec. 30, 2004), and 2003/0235582 (published Dec. 25, 2003) and Maloney et al. (2003), Cancer Res. 63:5073-5083, including but not limited to antibody EM164, resurfaced EM164, humanized EM164, huEM164 v1.0, huEM164 v1.1, huEM164 v1.2, and huEM164 v1.3 as described therein;

(vi) U.S. Pat. No. 7,037,498 (issued May 2, 2006), U.S. Publication Nos. 2005/0244408 (published Nov. 30, 2005) and 2004/0086503 (published May 6, 2004), and Cohen, et al. (2005), Clinical Cancer Res. 11:2063-2073, e.g., antibody CP-751,871, including but not limited to each of the antibodies produced by the hybridomas having the ATCC accession numbers PTA-2792, PTA-2788, PTA-2790, PTA-2791, PTA-2789, PTA-2793, and antibodies 2.12.1, 2.13.2, 2.14.3, 3.1.1, 4.9.2, and 4.17.3, as described therein;

(vii) U.S. Publication Nos. 2005/0136063 (published Jun. 23, 2005) and 2004/0018191 (published Jan. 29, 2004), including but not limited to antibody 19D12 and an antibody comprising a heavy chain encoded by a polynucleotide in plasmid 15H12/19D12 HCA (γ4), deposited at the ATCC under number PTA-5214, and a light chain encoded by a polynucleotide in plasmid 15H12/19D12 LCF (κ), deposited at the ATCC under number PTA-5220, as described therein; and

(viii) U.S. Publication No. 2004/0202655 (published Oct. 14, 2004), including but not limited to antibodies PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-11A5, PINT-11A7, PINT-11A12, PINT-12A1, PINT-12A2, PINT-12A3, PINT-12A4, and PINT-12A5, as described therein; each and all of which are herein incorporated by reference in their entireties, particularly as to the aforementioned antibodies, peptibodies, and related proteins and the like that target IGF-1 receptors;

B-7 related protein 1 specific antibodies, peptibodies, related proteins and the like (“B7RP-1,” also is referred to in the literature as B7H2, ICOSL, B7h, and CD275), particularly B7RP-specific fully human monoclonal IgG2 antibodies, particularly fully human IgG2 monoclonal antibody that binds an epitope in the first immunoglobulin-like domain of B7RP-1, especially those that inhibit the interaction of B7RP-1 with its natural receptor, ICOS, on activated T cells in particular, especially, in all of the foregoing regards, those disclosed in U.S. Publication No. 2008/0166352 and PCT Publication No. WO 07/011941, which are incorporated herein by reference in their entireties as to such antibodies and related proteins, including but not limited to antibodies designated therein as follow: 16H (having light chain variable and heavy chain variable sequences designated therein as, respectively, sequence identification number 1 and sequence identification number 7); 5D (having light chain variable and heavy chain variable sequences designated therein as, respectively, sequence identification number 2 and sequence identification number 9); 2H (having light chain variable and heavy chain variable sequences designated therein as, respectively, sequence identification number 3 and sequence identification number 10); 43H (having light chain variable and heavy chain variable sequences designated therein as, respectively, sequence identification number 6 and sequence identification number 14); 41H (having light chain variable and heavy chain variable sequences designated therein as, respectively, sequence identification number 5 and sequence identification number 13); and 15H (having light chain variable and heavy chain variable sequences designated therein as, respectively, sequence identification number 4 and sequence identification number 12), each of which is individually and specifically incorporated by reference herein in its entirety fully as disclosed in the foregoing publication;

IL-15 specific antibodies, peptibodies, and related proteins, and the like, such as, in particular, humanized monoclonal antibodies, particularly antibodies such as those disclosed in U.S. Publication Nos. 2003/0138421; 2003/023586; and 2004/0071702; and U.S. Pat. No. 7,153,507, each of which is incorporated herein by reference in its entirety as to IL-15 specific antibodies and related proteins, including peptibodies, including particularly, for instance, but not limited to, HuMax IL-15 antibodies and related proteins, such as, for instance, 146B7;

IFN gamma specific antibodies, peptibodies, and related proteins and the like, especially human IFN gamma specific antibodies, particularly fully human anti-IFN gamma antibodies, such as, for instance, those described in U.S. Publication No. 2005/0004353, which is incorporated herein by reference in its entirety as to IFN gamma specific antibodies, particularly, for example, the antibodies therein designated 1118; 1118*; 1119; 1121; and 1121*. The entire sequences of the heavy and light chains of each of these antibodies, as well as the sequences of their heavy and light chain variable regions and complementarity determining regions, are each individually and specifically incorporated by reference herein in its entirety fully as disclosed in the foregoing publication and in Thakur et al. (1999), Mol. Immunol. 36:1107-1115. In addition, description of the properties of these antibodies provided in the foregoing publication is also incorporated by reference herein in its entirety. Specific antibodies include those having the heavy chain of sequence identification number 17 and the light chain of sequence identification number 18; those having the heavy chain variable region of sequence identification number 6 and the light chain variable region of sequence identification number 8; those having the heavy chain of sequence identification number 19 and the light chain of sequence identification number 20; those having the heavy chain variable region of sequence identification number 10 and the light chain variable region of sequence identification number 12; those having the heavy chain of sequence identification number 32 and the light chain of sequence identification number 20; those having the heavy chain variable region of sequence identification number 30 and the light chain variable region of sequence identification number 12; those having the heavy chain sequence of sequence identification number 21 and the light chain sequence of sequence identification number 22; those having the heavy chain variable region of sequence identification number 14 and the light chain variable region of sequence identification number 16; those having the heavy chain of sequence identification number 21 and the light chain of sequence identification number 33; and those having the heavy chain variable region of sequence identification number 14 and the light chain variable region of sequence identification number 31, as disclosed in the foregoing publication. A specific antibody contemplated is antibody 1119 as disclosed in the foregoing U.S. publication and having a complete heavy chain of sequence identification number 17 as disclosed therein and having a complete light chain of sequence identification number 18 as disclosed therein;

TALL-1 specific antibodies, peptibodies, and the related proteins, and the like, and other TALL specific binding proteins, such as those described in U.S. Publication Nos. 2003/0195156 and 2006/0135431, each of which is incorporated herein by reference in its entirety as to TALL-1 binding proteins, particularly the molecules of Tables 4 and 5B, each of which is individually and specifically incorporated by reference herein in its entirety fully as disclosed in the foregoing publications;

Parathyroid hormone (“PTH”) specific antibodies, peptibodies, and related proteins, and the like, such as those described in U.S. Pat. No. 6,756,480, which is incorporated herein by reference in its entirety, particularly in parts pertinent to proteins that bind PTH;

Thrombopoietin receptor (“TPO-R”) specific antibodies, peptibodies, and related proteins, and the like, such as those described in U.S. Pat. No. 6,835,809, which is herein incorporated by reference in its entirety, particularly in parts pertinent to proteins that bind TPO-R;

Hepatocyte growth factor (“HGF”) specific antibodies, peptibodies, and related proteins, and the like, including those that target the HGF/SF:cMet axis (HGF/SF:c-Met), such as the fully human monoclonal antibodies that neutralize hepatocyte growth factor/scatter (HGF/SF) described in U.S. Publication No. 2005/0118643 and PCT Publication No. WO 2005/017107, huL2G7 described in U.S. Pat. No. 7,220,410 and OA-5d5 described in U.S. Pat. Nos. 5,686,292 and 6,468,529 and in PCT Publication No. WO 96/38557, each of which is incorporated herein by reference in its entirety, particularly in parts pertinent to proteins that bind HGF;

TRAIL-R2 specific antibodies, peptibodies, related proteins and the like, such as those described in U.S. Pat. No. 7,521,048, which is herein incorporated by reference in its entirety, particularly in parts pertinent to proteins that bind TRAIL-R2;

Activin A specific antibodies, peptibodies, related proteins, and the like, including but not limited to those described in U.S. Publication No. 2009/0234106, which is herein incorporated by reference in its entirety, particularly in parts pertinent to proteins that bind Activin A;

TGF-beta specific antibodies, peptibodies, related proteins, and the like, including but not limited to those described in U.S. Pat. No. 6,803,453 and U.S. Publication No. 2007/0110747, each of which is herein incorporated by reference in its entirety, particularly in parts pertinent to proteins that bind TGF-beta;

Amyloid-beta protein specific antibodies, peptibodies, related proteins, and the like, including but not limited to those described in PCT Publication No. WO 2006/081171, which is herein incorporated by reference in its entirety, particularly in parts pertinent to proteins that bind amyloid-beta proteins. One antibody contemplated is an antibody having a heavy chain variable region comprising sequence identification number 8 and a light chain variable region having sequence identification number 6 as disclosed in the foregoing publication;

c-Kit specific antibodies, peptibodies, related proteins, and the like, including but not limited to those described in U.S. Publication No. 2007/0253951, which is incorporated herein by reference in its entirety, particularly in parts pertinent to proteins that bind c-Kit and/or other stem cell factor receptors;

OX40L specific antibodies, peptibodies, related proteins, and the like, including but not limited to those described in U.S. Publication No. 2006/0002929, which is incorporated herein by reference in its entirety, particularly in parts pertinent to proteins that bind OX40L and/or other ligands of the OX40 receptor; and

Other exemplary proteins, including Activase® (alteplase, tPA); Aranesp® (darbepoetin alfa); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-α4ß7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Humatrope® (somatropin, Human Growth Hormone); Humira® (adalimumab); insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Soliris™ (eculizumab); pexelizumab (anti-05 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Neulasta® (pegylated filgastrim, pegylated G-CSF, pegylated hu-Met-G-CSF); Neupogen® (filgrastim, G-CSF, hu-MetG-CSF); Orthoclone OKT3® (muromonab-CD3, anti-CD3 monoclonal antibody); Procrit® (epoetin alfa); Remicade® (infliximab, anti-TNFα monoclonal antibody); Reopro® (abciximab, anti-GP IIb/IIIa receptor monoclonal antibody); Actemra® (anti-IL6 Receptor mAb); Avastin® (bevacizumab), HuMax-CD4 (zanolimumab); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 146B7-CHO (anti-IL15 antibody, see U.S. Pat. No. 7,153,507); Tysabri® (natalizumab, anti-α4integrin mAb); Valortim® (MDX-1303, anti-B. anthracis protective antigen mAb); ABthrax™; Vectibix® (panitumumab); Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2Ra mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNFα mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-α5β1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MY0-029); anti-GM-CSF Receptor mAb (CAM-3001); anti-HepC mAb (HuMax HepC); anti-IFNα mAb (MEDI-545, MDX-1103); anti-IGF1R mAb; anti-IGF-1R mAb (HuMax-Inflam); anti-IL12 mAb (ABT-874); anti-IL12/IL23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); anti-LLY antibody; BMS-66513; anti-Mannose Receptor/hCGβ mAb (MDX-1307); anti-mesothelin dsFv-PE38 conjugate (CAT-5001); anti-PD1mAb (MDX-1106 (ONO-4538)); anti-PDGFRα antibody (IMC-3G3); anti-TGFβ mAb (GC-1008); anti-TRAIL Receptor-2 human mAb (HGS-ETR2); anti-TWEAK mAb; anti-VEGFR/Flt-1 mAb; anti-ZP3 mAb (HuMax-ZP3); NVS Antibody #1; and NVS Antibody #2.

Also included can be a sclerostin antibody, such as but not limited to romosozumab, blosozumab, or BPS 804 (Novartis). Further included can be therapeutics such as rilotumumab, bixalomer, trebananib, ganitumab, conatumumab, motesanib diphosphate, brodalumab, vidupiprant, panitumumab, denosumab, NPLATE, PROLIA, VECTIBIX or XGEVA. Additionally, included in the device can be a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab), as well as molecules, variants, analogs or derivatives thereof as disclosed in the following patents or patent applications, each of which is herein incorporated by reference in its entirety for all purposes: U.S. Pat. No. 8,030,547, U.S. Publication No. 2013/0064825, WO2008/057457, WO2008/057458, WO2008/057459, WO2008/063382, WO2008/133647, WO2009/100297, WO2009/100318, WO2011/037791, WO2011/053759, WO2011/053783, WO2008/125623, WO2011/072263, WO2009/055783, WO2012/0544438, WO2010/029513, WO2011/111007, WO2010/077854, WO2012/088313, WO2012/101251, WO2012/101252, WO2012/101253, WO2012/109530, and WO2001/031007.

Also included can be talimogene laherparepvec or another oncolytic HSV for the treatment of melanoma or other cancers. Examples of oncolytic HSV include, but are not limited to talimogene laherparepvec (U.S. Pat. Nos. 7,223,593 and 7,537,924); OncoVEXGALV/CD (U.S. Pat. No. 7,981,669); OrienX010 (Lei et al. (2013), World J. Gastroenterol., 19:5138-5143); G207, 1716; NV1020; NV12023; NV1034 and NV1042 (Vargehes et al. (2002), Cancer Gene Ther., 9(12):967-978).

Also included are TIMPs. TIMPs are endogenous tissue inhibitors of metalloproteinases (TIMPs) and are important in many natural processes. TIMP-3 is expressed by various cells or and is present in the extracellular matrix; it inhibits all the major cartilage-degrading metalloproteases, and may play a role in role in many degradative diseases of connective tissue, including rheumatoid arthritis and osteoarthritis, as well as in cancer and cardiovascular conditions. The amino acid sequence of TIMP-3, and the nucleic acid sequence of a DNA that encodes TIMP-3, are disclosed in U.S. Pat. No. 6,562,596, issued May 13, 2003, the disclosure of which is incorporated by reference herein. Description of TIMP mutations can be found in U.S. Publication No. 2014/0274874 and PCT Publication No. WO 2014/152012.

Also included are antagonistic antibodies for human calcitonin gene-related peptide (CGRP) receptor and bispecific antibody molecule that target the CGRP receptor and other headache targets. Further information concerning these molecules can be found in PCT Application No. WO 2010/075238.

Additionally, bispecific T cell engager (BiTE®) antibody constructs, e.g. BLINCYTO® (blinatumomab), can be used in the device. Alternatively, included can be an APJ large molecule agonist e.g., apelin or analogues thereof in the device. Information relating to such molecules can be found in PCT Publication No. WO 2014/099984.

The term “bispecific” as used herein refers to an antibody construct which comprises at least a first binding domain and a second binding domain, wherein the first binding domain binds to one antigen or target, and the second binding domain binds to another antigen or target on the T cell. A preferred bispecific antibody construct according to the invention can also be defined as an antibody construct comprising a first binding domain which binds to a human antigen on the surface of a target cell and a second binding domain which binds to human CD3 on the surface of a T cell. Methods for preparing fused and operatively linked bispecific antibody constructs and expressing them in mammalian cells or bacteria are well-known in the art (e.g. WO 99/54440).

The invention provides a preferred embodiment wherein the bispecific antibody construct is in a format selected from the group consisting of (scFv)2, scFv-single domain mAb, diabodies and oligomers of any of those formats. According to a particularly preferred embodiment, the antibody construct of the invention is a bispecific single chain antibody construct, more preferably a bispecific single chain Fv (scFv).

In certain embodiments, the medicament comprises a therapeutically effective amount of an anti-thymic stromal lymphopoietin (TSLP) or TSLP receptor antibody. Examples of anti-TSLP antibodies that may be used in such embodiments include, but are not limited to, those described in U.S. Pat. Nos. 7,982,016, and 8,232,372, and U.S. Publication No. 2009/0186022. Examples of anti-TSLP receptor antibodies include, but are not limited to, those described in U.S. Pat. No. 8,101,182. In particularly preferred embodiments, the medicament comprises a therapeutically effective amount of the anti-TSLP antibody designated as A5 within U.S. Pat. No. 7,982,016.

Although the drug delivery devices, methods, and components thereof, have been described in terms of exemplary embodiments, they are not limited thereto. The detailed description is to be construed as exemplary only and does not describe every possible embodiment of the invention because describing every possible embodiment would be impractical, if not impossible. Numerous alternative embodiments could be implemented, using either current technology or technology developed after the filing date of this patent that would still fall within the scope of the claims defining the invention. For example, components described herein with reference to certain kinds of drug delivery devices, such as on-body injector drug delivery devices or other kinds of drug delivery devices, can also be utilized in other kinds of drug delivery devices, such as autoinjector drug delivery devices.

Those skilled in the art will recognize that a wide variety of modifications, alterations, and combinations can be made with respect to the above described embodiments without departing from the scope of the invention, and that such modifications, alterations, and combinations are to be viewed as being within the ambit of the inventive concept.

1. A system comprising:
a reservoir filled or fillable with a first drug product;
an administration member insertable into a patient and connected or connectable in fluid communication with the reservoir;
a fluid delivery system operable to deliver the first drug product from the reservoir to the patient via the administration member; and
a sensor operable to sense a biological condition of the patient.

2. The system of claim 1, comprising a controller configured to control operation of the fluid delivery system based on the sensed biological condition.

3. The system of claim 2, the controller being configured to operate the fluid delivery system to suspend, terminate, or throttle delivery of the first drug product to the patient based on the sensed biological condition.

4. The system of claim 2, the controller being configured to operate an output unit to notify the patient and/or a healthcare provider based on the sensed biological condition.

5. The system of claim 1, comprising a second reservoir filled or fillable with a second drug product, the controller being configured to operate the fluid delivery system to initiate delivery of the second drug product to the patient based on the sensed biological condition.

6. The system of claim 5, the controller being configured to operate the fluid delivery system to initiate delivery of the second drug product to the patient based on the sensed biological condition.

7. The system of claim 5, wherein the second drug product includes an agent for treating or managing a condition or syndrome induced by administration of the first drug product.

8. (canceled)

9. The system of claim 2, comprising:
a second sensor operable to sense a second biological condition of the patient; and
the controller being configured to either (a) or (b):
(a) operate the fluid delivery system to suspend, terminate, or throttle delivery of the first drug product to the patient in response to a determination that: (i) the sensed biological condition is within or outside of a predetermined range of values or is greater or less than a predetermined value, and/or (ii) the second sensed biological condition is within or outside of a second predetermined range of values or is greater or less than a second predetermined value, or
(b) operate the fluid delivery system to initiate delivery of the first drug product to the patient in response to a determination that: (i) the sensed biological condition is within or outside of a predetermined range of values or is greater or less than a predetermined value, and/or (ii) the second sensed biological condition is within or outside of a second predetermined range of values or is greater or less than a second predetermined value.

10. The system of claim 2, comprising:
a second reservoir filled or fillable with a second drug product;
the controller being configured to operate the fluid delivery system to initiate delivery of the second drug product to the patient in response to a determination that: (i) the sensed biological condition is within or outside of a predetermined range of values or is greater or less than a predetermined value, and/or (ii) the second sensed biological condition is within or outside of a second predetermined range of values or is greater or less than a second predetermined value.

11. The system of claim 10, the second drug product including an agent for treating or managing a condition or syndrome induced by administration of the first drug product.

12. (canceled)

13. The system of claim 2, the controller being configured to operate the fluid delivery system to initiate delivery of the first drug product to the patient in response to a determination that the sensed biological condition is within or outside of a predetermined range of values or is greater or less than a predetermined value.

14-36. (canceled)

37. The system of claim 1, wherein the fluid delivery system comprises a reconstitution subsystem and the first drug product comprises a lyophilized drug, the reconstitution subsystem being configured to reconstitute the lyophilized drug prior delivery to the patient via the administration member.

38. The system of claim 37, comprising a third reservoir filled or fillable with a diluent solution for reconstituting the lyophilized drug.

39. A method comprising:
operating a fluid delivery system to deliver a first drug product from a reservoir to a patient via an administration member; and
sensing, via a sensor, a biological condition of the patient while, before, and/or after the first drug product is delivered.

40. The method of claim 39, comprising operating the fluid delivery system to suspend, terminate, or throttle delivery of the first drug product to the patient based on the sensed biological condition.

41. (canceled)

42. The method of claim 39, comprising operating the fluid delivery system to initiate delivery of a second drug product from a second reservoir to the patient based on the sensed biological condition.

43. (canceled)

44. The method of claim 42, wherein the second drug product includes an agent for treating or managing a condition or syndrome induced by administration of the first drug product.

45. (canceled)

46. The method of claim 39, comprising:
sensing, via a second sensor, a second biological condition of the patient; and
at least one of (a) or (b):
(a) operating, automatically via the controller or manually, the fluid delivery system to suspend, terminate, or throttle delivery of the first drug product to the patient if: (i) the sensed biological condition is within or outside of the predetermined range of values or is greater or less than the predetermined value, and/or (ii) the second sensed biological condition is within or outside of a second predetermined range of values or is greater or less than a second predetermined value, or
(b) operating, automatically via the controller or manually, the fluid delivery system to initiate delivery of the first drug product to the patient if: (i) the sensed biological condition is within or outside of the predetermined range of values or is greater or less than the predetermined value, and/or (ii) the second sensed biological condition is within or outside of a second predetermined range of values or is greater or less than a second predetermined value.

47. The method of claim 46, comprising: operating, automatically via the controller or manually, the fluid delivery system to initiate delivery of a second drug product from a second reservoir to the patient if: (i) the sensed biological condition is within or outside of the predetermined range of values or is greater or less than the predetermined value, and/or (ii) the second sensed biological condition is within or outside of the second predetermined range of values or is greater or less than the second predetermined value.

48. The method of claim 47, wherein the second drug product includes an agent for treating or managing a condition or syndrome induced by administration of the first drug product.

49-51. (canceled)

52. The method of claim 39, comprising operating, automatically via the controller, an output unit to notify the patient and/or a healthcare provider that the sensed biological condition is within or outside of the predetermined range of values or is greater or less than the predetermined value.

53-65. (canceled)
